(19) United States
(12) Patent Application Publication
(10) Pub. No.: US 2025/0177735 A1
(43) Pub. Date: Jun. 5, 2025

(54) ELECTRIC-FIELD DIRECTED NERVE REGENERATION

(71) Applicant: UNIVERSITY OF SOUTHERN CALIFORNIA, Los Angeles, CA (US)

(72) Inventors: Kimberly GOKOFFSKI, Los Angeles, CA (US); Gianluca LAZZI, Los Angeles, CA (US)

(73) Assignee: UNIVERSITY OF SOUTHERN CALIFORNIA, Los Angeles, CA (US)

(21) Appl. No.: 18/844,153

(22) PCT Filed: Mar. 23, 2023
(86) PCT No.: PCT/US2023/016138

§ 371 Date: Sep. 5, 2024

Related U.S. Application Data
(60) Provisional application No. 63/323,029, filed on Mar. 23, 2022.

(51) Int. Cl. A61N 1/05; A61N 1/36

(52) U.S. Cl. CPC A61N 1/0551; A61N 1/36046; A61N 1/36103

(57) ABSTRACT

A retinal ganglion cell (RGC) stimulation system for an optic nerve. The system can comprise a ground electrode, a stimulation electrode, a voltage or current source connected to both the ground electrode and the stimulation electrode and configured to stimulate the stimulation electrode with an electrical waveform having a first voltage and a first current, and a controller connected to the voltage or current source and controlling the first voltage and the first current of the electrical waveform.

### CROSS-REFERENCE TO RELATED APPLICATIONS

This application claims the benefit of and priority to U.S. Provisional Application No. 63/323,029, filed on Mar. 23, 2022, which is herein incorporated by this reference in its entirety.

### STATEMENT AS TO FEDERALLY SPONSORED RESEARCH

This invention was made with government support under grants 1K08EY031797-01 from the National Institutes of Health (NIH) and OIA2121164 from the National Science Foundation (NSF). The government has certain rights in this invention.

### BACKGROUND

Significant interest exists in harnessing applied electrical fields into a therapy that can direct cellular processes such as nerve regeneration and wound healing. Although electric field stimulation with Direct Currents (DC) has shown promise to regenerate nerves, this cannot be employed in therapy since DC can introduce net charge into the tissue, thereby leading to tissue damage.

Therefore, in view of the above, there is a need for new electric field based therapies for cell regeneration.

### BRIEF DESCRIPTION OF THE DRAWINGS

To facilitate further description of the embodiments, the following drawings are provided in which:

FIG. 1 illustrates an exemplary system according to an embodiment;

FIG. 2 illustrates an exemplary waveform implemented in an embodiment;

FIG. 3 illustrates an exemplary heatmap summarizing data from exemplary embodiments;

FIG. 4 illustrates a graphical representation of nerve growth induced by different exemplary waveforms;

FIG. 5 illustrates a graphical representation of nerve growth induced by different exemplary waveforms; and

FIG. 6 illustrates results of exemplary treatment with systems described herein.

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

The terms “couple,” “coupled,” “couples,” “coupling,” and the like should be broadly understood and refer to connecting two or more elements mechanically and/or otherwise. Two or more electrical elements may be electrically coupled together, but not be mechanically or otherwise coupled together. Coupling may be for any length of time, e.g., permanent or semi-permanent or only for an instant. “Electrical coupling” and the like should be broadly understood and include electrical coupling of all types. The absence of the word “removably,” “removable,” and the like near the word “coupled,” and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “real-time” can, in some embodiments, be defined with respect to operations carried out as soon as practically possible upon occurrence of a triggering event. A triggering event can include receipt of data necessary to execute a task or to otherwise process information. Because of delays inherent in transmission and/or in computing speeds, the term “real time” encompasses operations that occur in “near” real time or somewhat delayed from a triggering event. In a number of embodiments, “real time” can mean real time less a time delay for processing (e.g., determining) and/or transmitting data. The particular time delay can vary depending on the type and/or amount of the data, the processing speeds of the hardware, the transmission capability of the communication hardware, the transmission distance, etc. However, in some embodiments, the time delay can be less than approximately one second, two seconds, five seconds, or ten seconds.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

### DETAILED DESCRIPTION

A number of embodiments can include a retinal ganglion cell (RGC) stimulation system for an optic nerve. The system can comprise a ground electrode; a stimulation electrode; a voltage or current source connected to both the ground electrode and the stimulation electrode and configured to stimulate the stimulation electrode with an electrical waveform having a first voltage and a first current; and a controller connected to the voltage or current source and controlling the first voltage and the first current of the electrical waveform.

Some embodiments can include A method of retinal ganglion cell (RGC) stimulation for an optic nerve. The method can comprise providing a ground electrode; providing a stimulation electrode; providing a voltage or current source connected to both the ground electrode and the stimulation electrode and configured to stimulate the stimulation electrode with an electrical waveform having a first voltage and a first current; and controlling by a controller connected to the voltage or current source, the first voltage and the first current of the waveform to generate a waveform, wherein the first voltage changes over time.

Various embodiments can include a system for electric-field directed nerve stimulation. The system can comprise a first electrode; a second electrode; a voltage or current source connected to both the first electrode and the second electrode and configured to stimulate the first electrode with an electrical waveform having a first voltage and a first current; and a controller connected to the voltage or current source and controlling the first voltage and the first current of the electrical waveform to induce a voltage differential across a nerve for regeneration, wherein the electrical waveform can comprise at least one of an asymmetric cathodic-first charge balanced biphasic waveform.

Further embodiments can include a system for electric-field directed nerve stimulation. The system can comprise a first electrode; a second electrode; a voltage or current source connected to both the first electrode and the second electrode and configured to stimulate the first electrode with an electrical waveform having a first voltage and a first current; and a controller connected to the voltage or current source and controlling the first voltage and the first current of the electrical waveform to induce a voltage differential across a nerve to promote cellular health, wherein the electrical waveform comprises a symmetric charge balanced biphasic waveform.

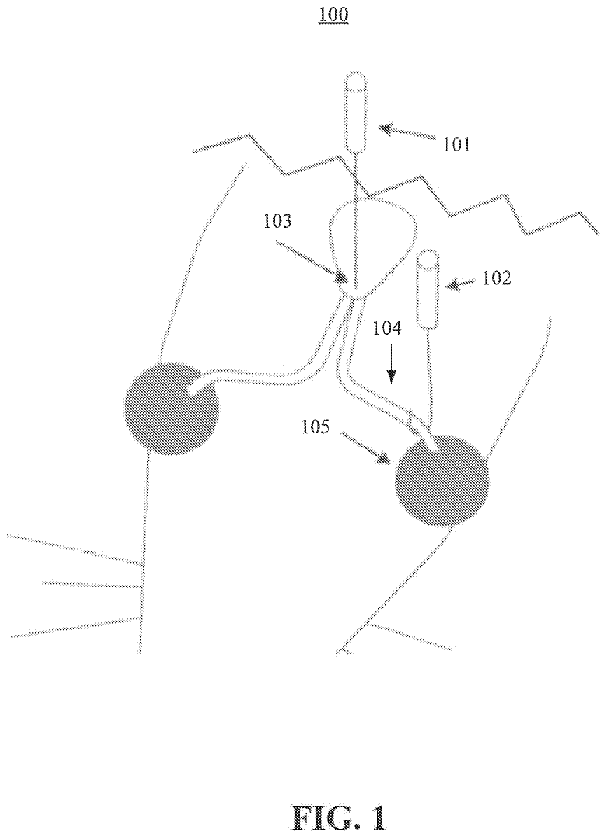

Turning to the drawings, FIG. 1 illustrates an exemplary embodiment of a system 100 for nerve regeneration. System 100 is merely exemplary and is not limited to the embodiments presented herein. System 100 can be employed in some different embodiments or examples not specifically depicted or described herein. In some embodiments, the elements of system 100 can be coupled in the arrangement presented. In other embodiments, the elements of system 100 can be coupled in any suitable arrangement. In still other embodiments, one or more of the elements of system 100 can be combined or omitted. In some embodiments, system 100 can comprise a ground electrode 101, a stimulation electrode 102, and/or a voltage or current source (not shown). Ground electrode 101 and a stimulation electrode 102 can be made from a variety of electrically conductive materials suitable for use on tissue. For example, ground electrode 101 and/or stimulation electrode 102 can be made from platinum and/or tungsten. In various embodiments, ground electrode 101 and stimulation electrode 102 can be made out of the same and/or different materials. In various embodiments, a platinum electrode can produce larger amplitudes along a nerve than a tungsten electrode.

In various embodiments, stimulation electrode can be wrapped around and/or inserted into a nerve (e.g., an optic nerve 104), thereby facilitating electrical coupling with nerve cells in the nerve. In various embodiments, stimulation electrode 102 can be placed on a first side of a nerve injury. For example, if optic nerve 104 is damaged, stimulating electrode 102 can be place behind an eye 105 of a patient. In some embodiments, stimulation electrode 102 can be placed near an axon terminal of a damaged nerve. In some embodiments, ground electrode 101 can be placed on a second side of a nerve injury and/or along a nerve tract. For example, if optic nerve 104 is damaged, ground electrode 101 can be placed along an optic nerve tract (e.g., at optic chasm 103). In some embodiments, ground electrode can be placed near a target area towards which nerve growth is desired. In various embodiments, ground electrode 101 and stimulation electrode 102 can be inserted into a patient in a stereotactic surgery.

In some embodiments, a voltage or current source can be electrically coupled to one or more of ground electrode 101 and a stimulation electrode 102. Generally speaking, a voltage or current source can be configured to produce a voltage gradient along a nerve. In various embodiments, a voltage gradient can be produced by electrifying (e.g., inducing a current into) stimulation electrode 102. In some embodiments, a voltage gradient can be used to direct neuronal (e.g., optic nerve 104) regeneration after an injury. For example, growth of retinal ganglion cells (RGCs) in an optic nerve can be directed toward ground electrode 101. In some embodiments, a voltage gradient can run from stimulation electrode 102 to ground electrode 101.

In various embodiments, system 100 can generate pulses characterized as working pulses which generate axonal regeneration, and recharging pulses which rebalance a charge in tissue. In various instances, a working pulse has a relatively lower amplitude and longer pulse width, while a recharging pulse has a relatively shorter pulse width and higher amplitude. As such, over time, the duration of tissue exposure to working pulses is greater than the duration of tissue exposure to recharging pulses, thereby causing a net axon growth associated with the working pulse to exceed the net axon growth associated with the recharging pulse. In this way, a sum of growth does not cancel. At the same time, because the recharging pulse has a higher amplitude, the net charge delivered across the tissue is null, as the recharging pulses balance the working pulses, ameliorating tissue damage.

In various embodiments, system 100 can generate positive and negative pulses with similar (e.g., same) length. In various instances, system 100 can generate positive and negative pulses with similar (e.g., same) amplitude. Thus, one may say that the system generates symmetric pulses. Rather than inducing net axon growth, such a system may promote cellular health in a cell of tissue exposed to the pulses. Such a system may encourage retinal ganglion cell survival. In further instances, the system may switch between producing asymmetric pulses and symmetric pulses. Thus, the system may alternately promote axon growth and also promote cellular health as directed by a controller. The controller may cause the system to produce different pulses of different durations and amplitudes responsive to an operator, sensors, and/or a preset program.

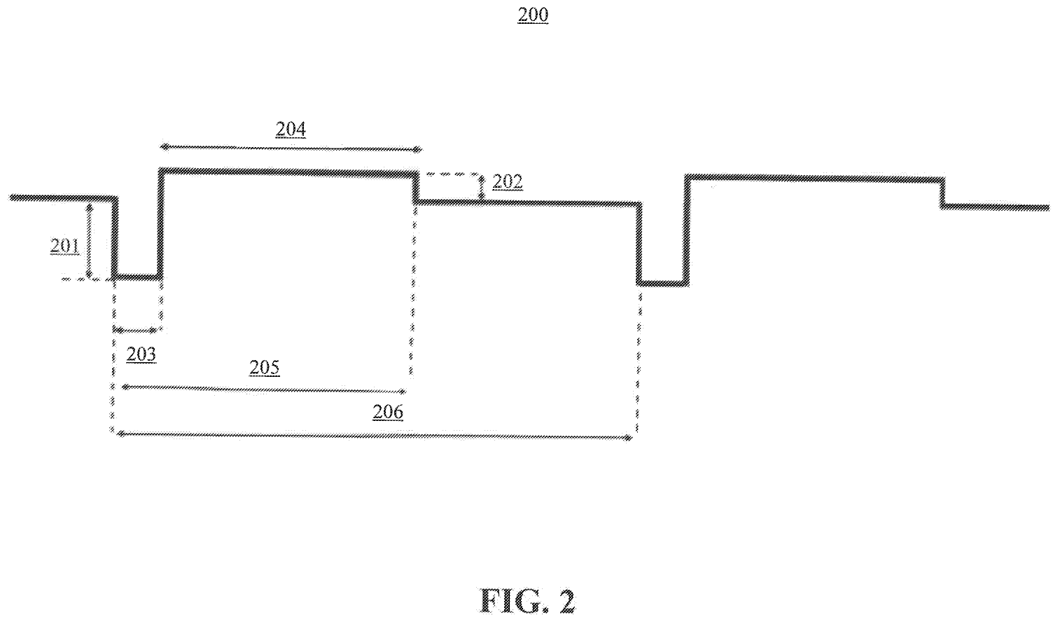

In some embodiments, a voltage or current source can be configured to generate a number of waveforms and/or electric fields configured to regenerate nerves. Turning now to FIG. 2, an exemplary waveform 200 is shown. Waveform 200 can be described by a cathodic voltage amplitude (V_{c}) 201, anodic voltage amplitude (V_{A}) 202, cathodic pulse width (PW_{C}) 203, anodic pulse width (PW_{A}) 204, overall width (W_{O}) 205, period 206, and phase duration ratio (PDR). In some embodiments, properties of waveform 200 can be calculated using the equations below:

$$\begin{matrix}{V}_{A} = \frac{{V}_{C}}{PDR} \\ PDR = \frac{{PW}_{A}}{{PW}_{C}}\end{matrix}$$

In an exemplary embodiment, a cathodic voltage amplitude can be set to 4 V with a pulse width of 0.1 ms and an anodic voltage amplitude was set to 1 V with a pulse width of 0.4 ms. In this exemplary embodiment, a PDR of 4 can be chosen due to the existence of a threshold electric field of 100 mV/mm for eliciting cathode directed growth in RGCs.

In some embodiments, a waveform 200 can be programmed and delivered by a controller (e.g., a computer system). For example, an Agilent 33220A Arbitrary Waveform Generator (AWG) and/or a STG 4008 Multi Channel Systems created by Harvard Bioscience, Inc. can be used to generate waveform 200. In various embodiments, a Keysight DSOX2014A oscilloscope can be used to record waveforms along a nerve while in a high-resolution acquisition modeIn order to record current passing through a nerve, a resistor (e.g., 10 Q) can be added in series to targeted tissue and a voltage across it can be read.

Returning now to FIG. 2, in some embodiments, waveform 200 can comprise an asymmetric charge balanced (ACB) waveform. In some embodiments, waveform 200 can comprise a biphasic voltage waveform where voltage is changed over time. In other words, waveform 200 can comprise both positive (i.e., anodic) pulses and negative (i.e., cathodic) pulses relative to a ground potential of ground electrode 101 (FIG. 1). In some embodiments, waveform 200 can comprise a cathodic first waveform or an anodic first waveform. Pulses can be structured in a number of ways in waveform 200. In some embodiments, positive pulses have can have a greater amplitude and a shorter duration than negative pulses. Negative pulses can have a greater amplitude and a shorter duration that positive pulses. In further embodiments, positive pulses and negative pulses can have a same pulse length in time and a same pulse amplitude. Positive and negative pulses in waveform 200 can have various functions. For example, positive pulses can stimulate nerve regeneration and negative pulses restore a charge balance across the nerve and/or individual nerve cells. In this way, nerve growth can be stimulated while preventing a damaging buildup of charge in the nerve or its cells.

In some embodiments, waveform 200 can be configured to induce a calcium influx in a stimulated nerve cell. Calcium influx in a nerve cell can induce cytoskeleton (e.g., actin) polymerization. In some embodiments, waveform 200 can induce asymmetric localization and/or activation of cell surface receptors and/or channels (e.g., voltage gated Ca²⁺ channels). In some embodiments, calcium influx can be higher in cathode oriented nerve cells than anode oriented nerve cells. In this way, waveform 200 can induce directional axonal growth in nerve cells.

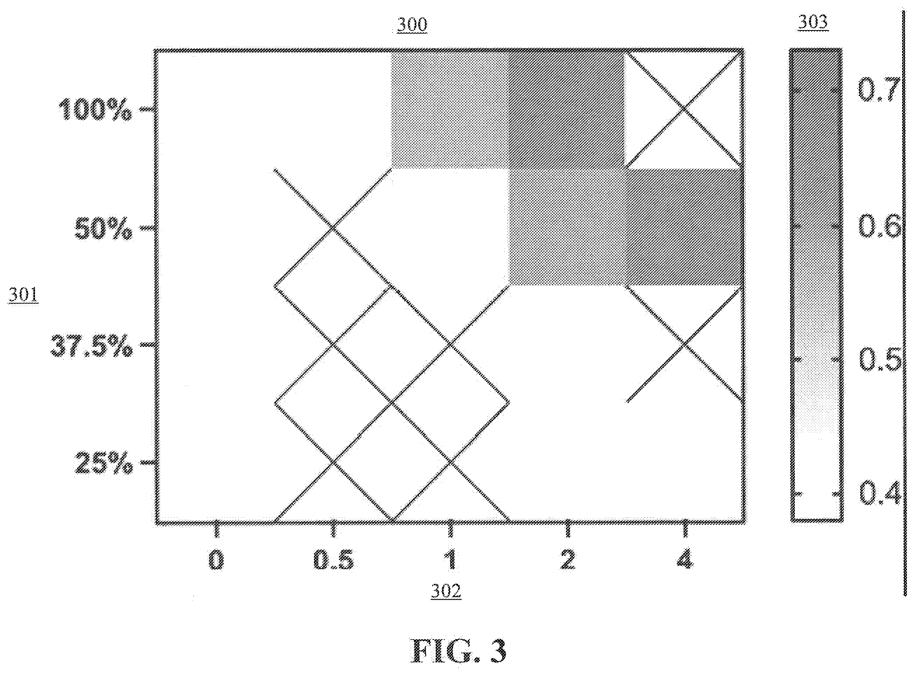

Turning ahead in the drawings, FIG. 3 displays an exemplary heatmap 300 summarizing data from exemplary monophasic pulses. In some embodiments, heatmap 300 can comprise y-axis 301, x-axis 302, and/or key 303. In further embodiments, heatmap 300 can define a parametric space for nerve growth inducing pulses in waveform 200 (FIG. 2). In this way, additional durations, structures, periods, amplitudes, widths, or other beneficial properties of waveform 200 (FIG. 2) can be obtained with little, if any, experimentation. In some embodiments, y-axis 301 can comprise a duty cycle. Generally speaking, a duty cycle can be understood as a ratio of a time a load or circuit is on compared to a time the load or circuit is off. In some embodiments, a circuit (e.g., a circuit created in system 100 (FIG. 1) can be considered off when the system implements a neutral polarity and/or a cathodic pulse. A circuit (e.g., a circuit created in system 100 (FIG. 1) can be considered off when the system implements an anodic pulse. In some embodiments, x-axis 302 can comprise a pulse amplitude in V/cm. Key 303 displays a value Q defining a ratio of nerve cells growing in a desired direction (e.g., towards optic chasm 103 (FIG. 3) as opposed to other directions. Cells in heatmap 300 with an X indicate parameters that were not tested.

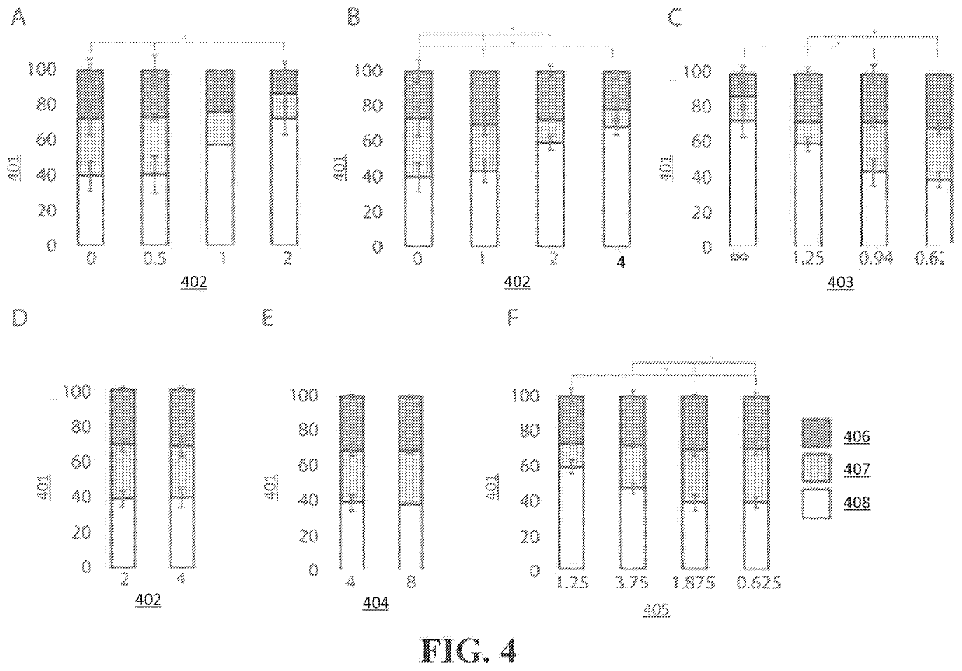

Turning now to FIG. 4, a graphical representation of nerve growth induced by different exemplary waveforms is shown. In some embodiments, axis 401 can comprise a percent axon growth, axis 402 can comprise a voltage gradient in V/cm, axis 403 can comprise a pulse width in seconds, axis 404 can comprise a current duration in hours, axis 405 can comprise interpulse delay in seconds, key 406 can comprise perpendicular nerve growth, key 407 can comprise anode directed nerve growth, and key 408 can comprise cathode directed nerve growth. As can be seen in FIG. 4A, DC experiments demonstrated that pulse amplitude plays an important role in determining whether a waveform will be effective at directing axon growth. As seen in FIG. 4B, purified RGCs directed their axons to grow towards a cathode when exposed to a monophasic waveform with a similar pulse width (2 V/cm, 400 mHz, 50% duty cycle). FIG. 4C, in turn, shows that decreases in pulse width below 1.25 secs were associated with proportional decreases in nerve growth. As can be seen in FIG. 4D, increasing an amplitude was unable to compensate for a loss of effect experienced from decreasing a pulse width. Even doubling an experimental timeline, as shown in FIG. 4E, or halving the interpulse interval (FIG. 4F, two right bars) was unable to compensate for a shorter pulse width. Conversely, increasing an interpulse interval for effective waveforms did not neutralize the effect of a waveform on directing RGC axon growth (FIG. 4F, two left bars). These experiments show that a pulse width threshold can be used to effectively direct axon growth. Pulse amplitude can also be used to determine a threshold effect on nerve growth. In some embodiments, doubling a stimulation amplitude from 2 V/cm to 4 V/cm did not result in a doubling of percent axons that grew towards a cathode (FIG. 4B). Therefore, when above a certain threshold, increasing stimulation amplitude does not have an additive effect on directing axon growth.

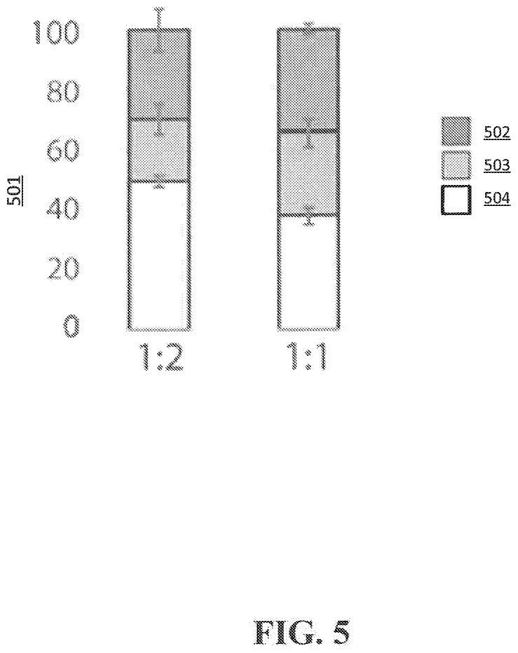

Turning ahead in the drawings, FIG. 5 illustrates a graphical representation of nerve growth induced by different exemplary waveforms. In some embodiments, axis 501 can comprise a percent axon growth, key 502 can comprise perpendicular nerve growth, key 503 can comprise anode directed nerve growth, and key 50 4can comprise cathode directed nerve growth. In some embodiments, FIG. 5 can show that pairing monophasic waveforms that are effective at directing axon growth with monophasic waveforms that are ineffective, but of the opposite polarity, can cause RGC axon growth. Therefore, FIG. 5 displays results from an embodiment where a 2 V/cm, 400 mHz, 50% DC anodic working pulse with a −4 V/cm, 400 mHz, 25% DC cathodic recharging pulse (i.e. a 1:2 ACB). FIG. 5 shows that this waveform can be used to direct cathodic growth of purified RGC cells over controls. When a pulse amplitude to pulse width ratio was set to 1:1 (e.g., as in a traditional AC current), axonal galvanotaxis did not occur.

In various embodiments, the system includes a voltage source that has an active circuit. In some embodiments, an active circuit may increase a voltage between a ground electrode and a stimulation electrode while limiting a current between the ground electrode and stimulation electrode. In some embodiments, an active circuit may be a negative-equivalent resistance circuit. In various embodiments, a voltage source may have a capacitive circuit to increase a voltage between the ground electrode and stimulation electrode while limiting a current between the ground electrode and stimulation electrode. In some embodiments, a voltage and/or current source can have negative resistance. For example, a voltage or current source can comprise a non-Foster circuit. Generally speaking, a non-Foster circuit can be described as an active circuit with an equivalent negative resistance when attached in series to a passive system. In this way, an interface impedance between an electrode (e.g., stimulation electrode 102 FIG. 1) and tissue (e.g., optic nerve 104 (FIG. 1)) can be manipulated. In further embodiments, a non-Foster circuit can increase a voltage gradient along a nerve by increasing a capacitive conduction between a stimulation and ground electrode. Increasing capacitive conduction in a nerve circuit can then reduce a faradaic conduction of ions across an electrode-electrolyte interface, thereby preventing a reaction that causes electric field induced tissue damage. In some embodiments, a non-Foster circuit can be configured to increase a voltage difference between a stimulation electrode 102 (FIG. 1) and ground electrode 101 (FIG. 1) by 40% while delivering a same input current as a comparable Foster circuit.

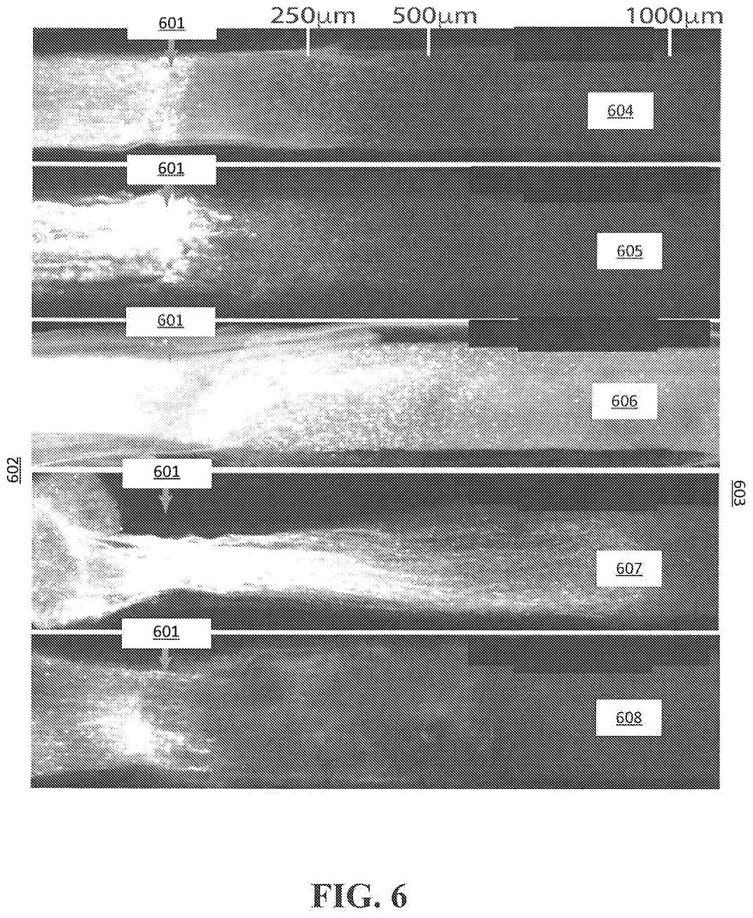

In some embodiments, waveform 200 (FIG. 2) implemented on system 100 (FIG. 1) can be used to treat nerve damage in a patient. Turning ahead in the drawings, FIG. 6 displays exemplary results of treatment using waveforms similar to waveform 200 (FIG. 2) implemented on systems similar to system 100 (FIG. 1) using parameters derived from heat map 300 (FIG. 3) in a rat model. In some embodiments, injury site 601 can comprise a location where a nerve was severed and/or damaged, lefthand side 602 can comprise a direction of an eye, righthand side 603 can comprise a direction towards a brain, nerve 604 can comprise an untreated nerve, and nerves 605-608 can comprise nerves treated with waveforms similar to waveform 200 (FIG. 2) using different parameters derived from heat map 300 (FIG. 3). In some embodiments, pulse widths ranging between 100 to 400 μseconds, amplitudes ranging between −400 to +400 μAmp, and/or a duty cycle of 50% can be used. As can be seen in FIG. 6, stimulation using waveforms similar to waveform 200 (FIG. 2) can cause nerve regeneration across a crush site. In some embodiments, treatment with waveforms similar to waveform 200 (FIG. 2) for 30 days can regenerate a nerve and restore eyesight. For example, 67% of rats whose optic nerves were crushed and then regenerated using electric field stimulation detected a visual cliff while no rats in an untreated group detected the visual cliff.

Although systems and methods for electric field directed nerve regeneration have been described with reference to specific embodiments, it will be understood by those skilled in the art that various changes may be made without departing from the spirit or scope of the disclosure. Accordingly, the disclosure of embodiments is intended to be illustrative of the scope of the disclosure and is not intended to be limiting. It is intended that the scope of the disclosure shall be limited only to the extent required by the appended claims. For example, to one of ordinary skill in the art, it will be readily apparent that any element of FIGS. 1-6 may be modified, and that the foregoing discussion of certain of these embodiments does not necessarily represent a complete description of all possible embodiments.

All elements claimed in any particular claim are essential to the embodiment claimed in that particular claim. Consequently, replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

What is claimed is:

1. A retinal ganglion cell (RGC) stimulation system for an optic nerve, the system comprising:
a ground electrode;
a stimulation electrode;
a voltage or current source connected to both the ground electrode and the stimulation electrode and configured to stimulate the stimulation electrode with an electrical waveform having a first voltage and a first current; and
a controller connected to the voltage or current source and controlling the first voltage and the first current of the electrical waveform.

2. The RGC stimulation system according to claim 1, wherein the electrical waveform is an asymmetric charge balanced biphasic waveform configured to promote neuronal regeneration of a retinal ganglion cell axon, wherein the first voltage changes over time.

3. The RGC stimulation system according to claim 1, wherein the RGC stimulation system promotes neuronal regeneration of a retinal ganglion cell axon in the optic nerve.

4. The RGC stimulation system according to claim 1, wherein the RGC stimulation system promotes cell health in the optic nerve.

5. The RGC stimulation system according to claim 1, wherein the ground electrode is connected to an optical nerve at an optic tract, and wherein the stimulation electrode is connected to the optical nerve, wherein a voltage gradient is induced along the optic nerve to direct neuronal regeneration of a retinal ganglion cell axon between the stimulation electrode and the ground electrode.

6. The RGC stimulation system of claim 1, wherein the ground electrode and the stimulation electrode are selected from a group consisting of: (i) the ground electrode and the stimulation electrode are both platinum, (ii) the ground electrode and the stimulation electrode are both tungsten, and (iii) the ground electrode is tungsten and the stimulation electrode is platinum.

7. The RGC stimulation system of claim 1, wherein the electrical waveform comprises both positive pulses and negative pulses relative to a ground potential of the ground electrode, wherein the positive pulses have greater amplitude and shorter duration and the negative pulses have lower amplitude and longer duration.

8. The RGC stimulation system of claim 1, wherein the electrical waveform comprises both positive pulses and negative pulses relative to a ground potential of the ground electrode, wherein the negative pulses have greater amplitude and shorter duration and the positive pulses have lower amplitude and longer duration.

9. The RGC stimulation system of claim 1, wherein the electrical waveform comprises both positive pulses and negative pulses relative to a ground potential of the ground electrode, wherein the positive pulses stimulate neuronal regeneration of a retinal ganglion cell axon and the negative pulses restore a charge balance.

10. The RGC stimulation system of claim 1, wherein the electrical waveform comprises both positive pulses and negative pulses relative to a ground potential of the ground electrode, wherein the positive pulses and the negative pulses are of a same pulse length in time and a same pulse amplitude, wherein a combination of the positive pulses and the negative pulses promotes cellular health of a cell of the optic nerve.

11. The RGC stimulation system of claim 1, wherein the electrical waveform stimulates RGC axon growth toward an electrode of the ground electrode and the stimulation electrode has a positive voltage relative to the ground electrode.

12. The RGC stimulation system according to claim 1, wherein the electrical waveform is an asymmetric cathodic-first charge balanced biphasic waveform.

13. A method of retinal ganglion cell (RGC) stimulation for an optic nerve comprising:
providing a ground electrode;
providing a stimulation electrode;
providing a voltage or current source connected to both the ground electrode and the stimulation electrode and configured to stimulate the stimulation electrode with an electrical waveform having a first voltage and a first current; and
controlling by a controller connected to the voltage or current source, the first voltage and the first current of the electrical waveform to generate a waveform, wherein the first voltage changes over time.

14. The method of RGC stimulation according to claim 13, wherein generating the waveform comprises:
generating both positive pulses and negative pulses relative to a ground potential of the ground electrode;
stimulating, by the positive pulses, neuronal regeneration of a retinal ganglion cell axon; and
restoring, by the negative pulses, a charge balance.

15. The method of RGC stimulation according to claim 14, wherein the negative pulses have greater amplitude and shorter duration and the positive pulses have lower amplitude and longer duration.

16. The method of RGC stimulation according to claim 13, where the waveform is an asymmetric charge balanced biphasic waveform to promote neuronal regeneration of a retinal ganglion cell axon in the optic nerve.

17. The method of RGC stimulation according to claim 13, where the waveform is a symmetric charge balanced biphasic waveform to promote cell health in the optic nerve.

18. The method of RGC stimulation according to claim 13, wherein providing the voltage or current source further comprises providing an active circuit and the method of RGC stimulation further comprises:
increasing, by the active circuit, the first voltage between the ground electrode and the stimulation electrode; and
limiting, by the active circuit, the first current between the ground electrode and the stimulation electrode.

19. A system for electric-field directed nerve stimulation comprising:
a first electrode;
a second electrode;
a voltage or current source connected to both the first electrode and the second electrode and configured to stimulate the first electrode with an electrical waveform having a first voltage and a first current; and
a controller connected to the voltage or current source and controlling the first voltage and the first current of the electrical waveform to induce a voltage differential across a nerve for regeneration,
wherein the electrical waveform comprises at least one of an asymmetric cathodic-first charge balanced biphasic waveform.

20. A system for electric-field directed nerve stimulation comprising:
a first electrode;
a second electrode;
a voltage or current source connected to both the first electrode and the second electrode and configured to stimulate the first electrode with an electrical waveform having a first voltage and a first current; and
a controller connected to the voltage or current source and controlling the first voltage and the first current of the electrical waveform to induce a voltage differential across a nerve to promote cellular health,
wherein the electrical waveform comprises a symmetric charge balanced biphasic waveform.
